(19) United States
(12) Patent Application Publication
(10) Pub. No.: US 2017/0132383 A1
(43) Pub. Date: May 11, 2017

(54) SYSTEMS AND METHODS FOR AUTOMATED RULE GENERATION AND DISCOVERY FOR DETECTION OF HEALTH STATE CHANGES

(71) Applicant: SENTRIAN, INC., Aliso Viejo, CA (US)

(72) Inventors: Lance Jonathan Myers, Aliso Viejo, CA (US); Jack Kreindler, London (GB); Martin S. Kohn, Aliso Viejo, CA (US)

(21) Appl. No.: 14/937,484

(22) Filed: Nov. 10, 2015

(51) Int. Cl. G06F 19/00; G06F 3/0482; G08B 29/18; G06T 11/20; G08B 21/02; G06F 17/24; G06F 3/0484

(52) U.S. Cl. CPC G06F 19/3406; G06F 17/241; G06F 3/0482; G06F 3/04847; G06T 11/206; G08B 21/02; G08B 29/185; A61B 5/021

(57) ABSTRACT

A health-state detection system configured to receive medical measurement data for one or more patients; a care manager interface for communicating with care managers; a user interface for receiving rules related to each type of medical measurement data; processors for parametrize the rules to generate a set of parameterized rules that define shifts, drifts and trends in the medical measurement data, determine a baseline for each type of medical measurement data, execute the parameterized rules for newly received medical measurement data against the baseline to detect shifts, drifts or trends in the medical measurement data, determine the magnitudes and directions of the detected shifts, drifts or trends, correlate the magnitudes and directions of the shifts, drifts, or trends detected with changes in the health state of one or more patients, and provide customizable notifications, alerts to care managers based at least in the past on the detected health state changes.

### BACKGROUND

Humans are prone to unexpected disease or sub-optimal wellness. It is possible to instrument humans with objective measurement sensors, and/or use sensor to passively acquire biometric or behavioral measurements, and/or collect quantifiable environmental and subjective observations, which generate data that can be used to improve well-being and predict future health compromise.

Biometric sensors may be used by patients in their home or community setting and may be used in a continuous, ambulatory manner or used intermittently under controlled or non-controlled conditions. Data from these sensors may be wirelessly transmitted to a central processor and it is possible to obtain on-going data feeds from these measurements at a variety of different rates.

But the true “state” of an individual is often not readily observed or measured, and changes in state of measurable parameters or observations are used to infer changes in internal state. There are a plethora of conventional sensors that may be used to acquire some physiological or psychological parameter that may be tracked on their own or together to determine changes in parameter, which in turn infer changes in health state.

Existing monitoring systems allow for operators to set change detection thresholds or rules based upon their experience, knowledge, intuition and evidence in order to automatically generate notifications when computer programs evaluate these rules against newly acquired incoming measurement data. These systems often are not accurate and are prone to false alarms and missed events. Such systems are also static in that they do not allow for automatic updates or generation of new rules or thresholds to improve detection accuracy. They also do not consider any feedback of target events or health state-changes which can be used to update the detection rules and thus improve accuracy toward the desired targets.

### SUMMARY

According to one aspect, a health-state detection system that has: a communications interface system for receiving medical measurement data pertaining to one or more patients; a care manager interface system for communicating with one or more care managers of the patients; and a rules engine system coupled to the communications interface system and to the care manager interface. The rules engine system is programmed to: first execute parameterized rules that detect shifts, drifts or trends in medical measurement data, wherein the rule parameters determine the magnitudes and directions of the shifts, drifts or trends to be detected; then detect changes in the health state of one or more patients in dependence on shifts, drifts, or trends detected by the parameterized rules in the medical measurement data; and finally provide customizable notifications, alerts or alarms to care managers over the care manager interface system in dependence on the detected health state changes. Optionally, the system can also include a measurement database storing historic medical measurement data received over the communication interface system, wherein the medical measurement data comprises one or more of biometric data, clinical data, laboratory data, environmental data, and observation data.

Accordingly to another aspect, a health-state detection method that: executes parameterized rules that detect shifts, drifts or trends in medical measurement data from one or more patients, wherein the parameters determine the magnitudes and directions of the shifts, drifts or trends to be detected; detects changes in the health state of one or more patients in dependence on shifts, drifts, or trends detected by the parameterized rules in the medical measurement data; and provides customizable notifications, alerts or alarms to care managers over the care manager interface in dependence on the detected health state changes. Optionally, the preferred second embodiment stores historic medical measurement data received over the communication interface in a measurement database, wherein the medical measurement data comprises one or more of biometric data, clinical data, laboratory data, environmental data, and observation data.

### BRIEF DESCRIPTION OF THE DRAWINGS

FIG. 1 illustrates one example process for detecting shifts, drifts and trends in data in accordance with one embodiment;

FIG. 2 illustrates another example process for detecting shifts, drifts and trends in data in accordance with one embodiment;

FIG. 3 illustrates an example adaptive windowing method that can be used in the processes of FIGS. 1 and 2;

FIG. 4 illustrates an example of a rule builder or rule configuration that can be implemented for the adaptive windowing method of FIG. 3;

FIG. 5 illustrates an example of how a decision tree can learn new rules and rule parameters for the adaptive windowing method of FIG. 3;

FIG. 6 illustrates an example of how rules generated by implementation of the process of FIGS. 1-5 can be presented to the user as recommendations;

FIG. 7 is a block diagram illustrating an example system that can be configured to implement the process of FIGS. 1-6; and

FIG. 8 is a diagram illustrating a processing system that can be used to implement, or included in the system of FIG. 7 and on which one or more of the processes described herein may be executed, according to an embodiment.

### DETAILED DESCRIPTION

Embodiments of the systems and methods described herein are directed to determining changes in health state by inference from changes in measurement data. Measurement data can be obtained from any one or more of biometric sensing devices, observational data feeds, subjective assessments, environmental data, health record data, laboratory measurements, imaging data, and so forth. The algorithms that detect changes in health state can be formatted as a set of heuristics that describe rules which are automatically evaluated against acquired measurement data.

Examples of biometric data include heart rate, respiration rate, weight, blood pressure, patient movements, and so forth. Examples of observational data include weight, swelling, urine output, and so forth. Examples of subjective assessments include easy tiring, labored breathing, difficulty walking, in pain, and so forth. Examples of environmental data include temperature, humidity, and so forth. Examples of health record data include past and current diagnoses, subjective and observational data, visits to the doctor, hospital admissions, and so forth. Examples of imaging data include findings on X-ray, CT scan or MRI such as presence of a fracture, or a mass, or fluid, and so forth.

Regular or irregular, longitudinal measurements obtained from these sensors may be integrated with other health state measurements obtained from the environment, human observation, electronic health records, claims data, laboratory data and/or imaging data. Any or all of these measurements may be continuously analyzed by automated algorithms in order to detect any desired changes in health state.

Changes in physiological and/or psychological health state may not immediately manifest in noticeable symptoms that could be reported or acted upon. However, the ability to efficiently distinguish natural fluctuations from symptomatic tendencies can allow for early warnings and appropriate responses. The means to identify and detect relevant changes in physiological and/or psychological status shortly after the occurrence of the change is of significant advantage in the management of health and wellness.

Perturbations to an individual's physiological and/or psychological dynamic processes can occur due to a variety of factors. These include drug intake, compensatory homeostatic mechanisms, disease onset or progression, changing environmental factors, trauma, ageing etc. These perturbations will result in either an abrupt shift or a gradual drift, which may trend in a particular direction. The system and methods described herein are intended to detect any shift, drift or trend onset at an early time point so that appropriate counter-measures may be taken.

Changes to physiological and/or psychological processes may occur in an acute setting such as monitoring after surgery in an ICU, or they may occur over an extended time period, spanning several days, weeks, months or years. Changes may occur in the measured subjects home or community setting, or in any setting where measurements may be made on a regular basis. This invention is applicable to both acute and long term change detection, with a focus or preference for long-term monitoring. Longitudinal monitoring allows for the establishment of an appropriate, individual specific, baseline, against which the shift or drift may be measured.

### Pre-Processing

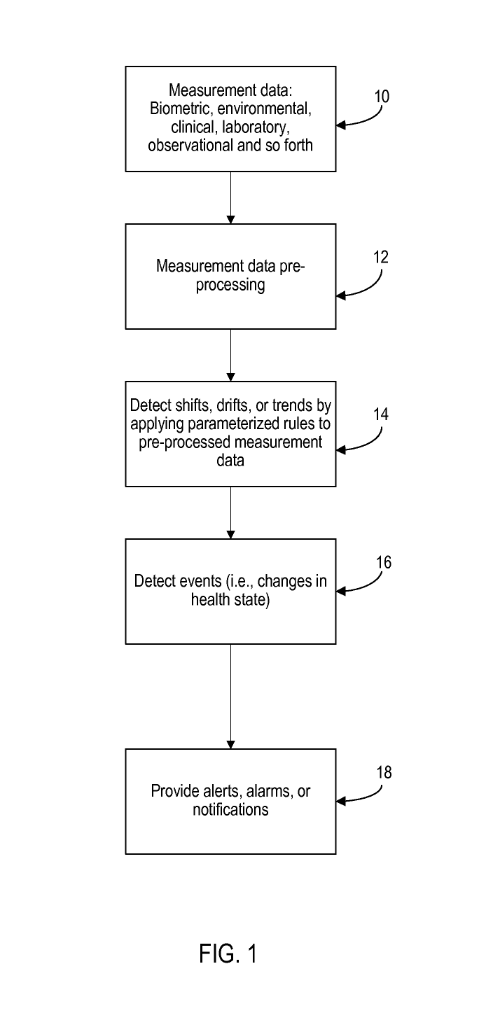

FIG. 1 is a flow chart that illustrates an example process for detecting events in a measured data in accordance with one embodiment. In this embodiment, there is no rule discovery or parameter learning. But as will be seen below, the systems and methods described herein can be implement with the assistance of rule discovery and parameter learning. In the process of FIG. 1, incoming measurement data received in step 10 are optionally pre-processed in step 12. Then shifts, drifts or trends are detected in step 14 by applying configurable or programmable rules to the pre-processed data. The detected shifts, drifts or trends are optionally combined and changes in health state are inferred in step 16. Changes in health state, or the changed health state itself, are referred to herein as “health events.” Finally, alerts, alarms or notifications can be provided, in step 18, to the system's users from the changes in health state, or events.

The measurement received in step 10 can include biometric data, such as described above; environmental data, such as described above; laboratory data; observational data, etc. Each type data can then be appropriately pre-processed to extract more relevant features and to discard less relevant features. For example, pre-processing substantially continuous data can include many possible digital signal processing steps that smooth, transform, aggregate or otherwise manipulate the incoming data feeds. For example, data can be smoothed by low pass filtering and high pass filtering can be used to emphasize transitions in the data. Data can also be transformed and then replaced by lower order transform coefficients while the higher order coefficients are discarded (or vice versa).

Pre-processing categorical data, i.e., data taking values in a discrete finite set, can be preprocessed by counting. For Example, the number of occurrences greater than a threshold in finite intervals can be counted and passed along. Alternatively, the data can be made continuous by making the categorical values coefficients of a series of continuous functions.

It can be seen how this preprocessing can be controlled by parameters: digital filter coefficients, number of transform coefficients retained, threshold values, counting interval, etc. These parameters can be tuned/learned as will be subsequently described.

The result of any pre-processing is a set of transformed measurements that are provided for further processing or analysis. The data can be pre-processed as soon as acquired or after a specified latency.

### Shift, Drift and Trend Detection

After pre-processing in step 12, shift, drift or trend detection can occur in step 14. First, a baseline is established for each type of measurement data or combination(s) of types. The baseline can be a fixed value, or a statistical summary of several measured parameters (i.e., average, median and standard deviation). The baseline can be extended or updated as new data are acquired. The baseline can be configured or programmed by a user of the system, or it may be pre-determined.

Second, after baseline establishment and as further data arrives, the new data is tracked against the relevant baseline to determine if significant shifts, drifts or trends away from the baseline are occurring. The presence or absence of detected shifts, drifts or trends are, preferably, the elementary data items that are combined and analyzed in step 16 to infer health events.

The determination that a shift, trend or drift occurs, or does not occur, in a particular type of measurement data is the raw material from which events, i.e., changes in health states, are detected in subsequent step 16.

Concerning shift detection, new measurements from each sensor feed or combination of feeds are tested against their matched baselines to assess if there is a statistically significant shift. From a statistical perspective, the null hypothesis is that the previously seen values or baseline values and the currently observed values both come from the same distribution. The alternative hypothesis is that they are generated from different distributions. A variety of established statistical tests can be used to compare the current points against the baseline data to evaluate the null hypothesis. These tests include simple threshold crossings, student t-tests, and non-parametric tests such as the Mann-Whitney U-test or the Rank-Sum test. Tests can be conducted on single parameters or multivariate tests can be used on a collection of parameters. Alternatively, the parameters can first be combined into a composite index prior to testing.

An alternative approach for shift detection is to frame the problem as novelty detection. Here a boundary is drawn in N-dimensional feature space around the baseline data. If a new point occurs outside this boundary within a given margin or tolerance, a shift is deemed to have occurred. Examples of novelty detection are simple threshold crossings, one-class support vector machines (SVM), k-nearest neighbor, neural networks and other known techniques in the statistical machine learning art.

Concerning drift or trend detection, such detection is often more difficult to assess. Each approach used with the systems and methods described herein can operate on univariate data, multivariate data, or a composite index. Multiple tests can be performed and an alert triggered if one or more tests agree to a drift or trend detection. In drift/trend change detection, the baseline data is updated as each new processed data feed is acquired. Weighted time windows can be used to update the baseline data.

The statistical process control drift detection method monitors the error between the new sample and an estimate of the new data. Two thresholds are described. When the error exceeds the first (warning) threshold, the system enters a warning mode and stores the date and time this occurred. If the error drops below this threshold, the warning state is cancelled; however, if in a following sequence, the error continues to increase and then exceeds a second threshold, a drift change is declared and an alert triggered.

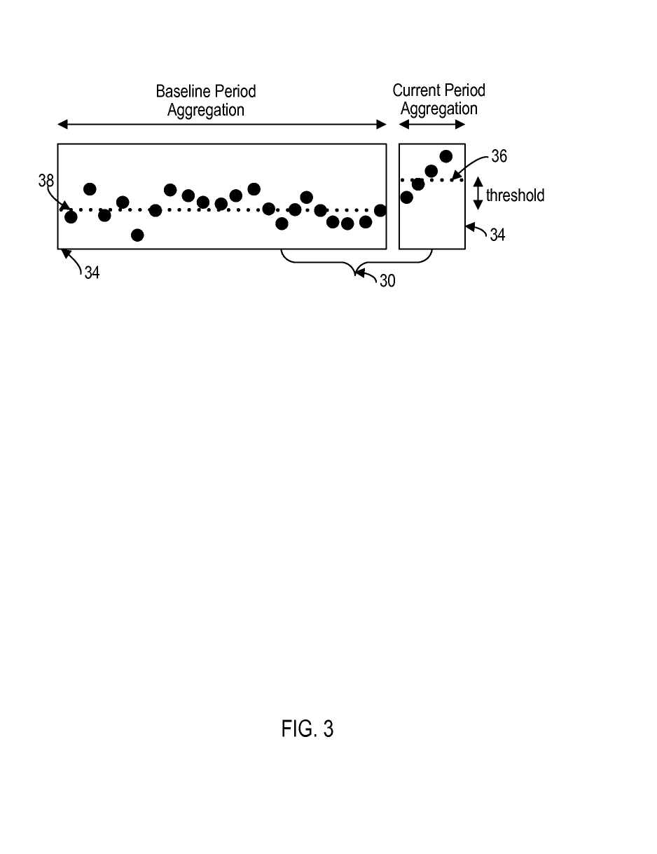

The adaptive windowing method (see Example and FIG. 3) employs a sliding window containing the most recently received data points and then compares the distribution on two sub windows of this sliding window. Whenever two large enough sub-windows exhibit statistical significant differences, an alert is triggered. Standard statistical hypothesis tests, or other basic threshold comparisons may be used in each sub window. This method is described in more detail in A. Bifet and R. Gavald'a. Learning from Time-Changing Data with Adaptive Windowing. Proceedings of the 7th SIAM International Conference on Data Mining, Minneapolis, Minn., USA, 2007 and is incorporated herein by reference.

The fixed cumulative windows model is similar to the above method but only updates a new current window and compares this distribution against the baseline or reference distribution. A preferred embodiment is to use the Kullback-Leibler divergence (KLD) to define a threshold for change detection decisions based on the asymmetry of the data.

The Page-Hinkley test (PHT) uses a cumulative variable defined as the cumulative difference between the observed data and its mean, less a pre-defined magnitude threshold. The mean is taken from some point in the past until the current point in time. The time of the minimum value of this cumulative sum is computed and the difference between the cumulative sum and the mean is computed. When this difference exceeds a given threshold, a change in the distribution is assigned. Controlling the detection threshold makes it possible to establish a trade-off between false positives and false negatives.

In accordance with certain embodiments, further analysis of changes or drifts in baseline condition can be obtained by examining more than one of the above methods and using weighted decision rules to determine overall change. In other embodiments, methods of combining several algorithms into a single meta-decision can use a first algorithm to trigger computation of one or more additional algorithms before assigning a final decision as to whether a significant change or drift has occurred.

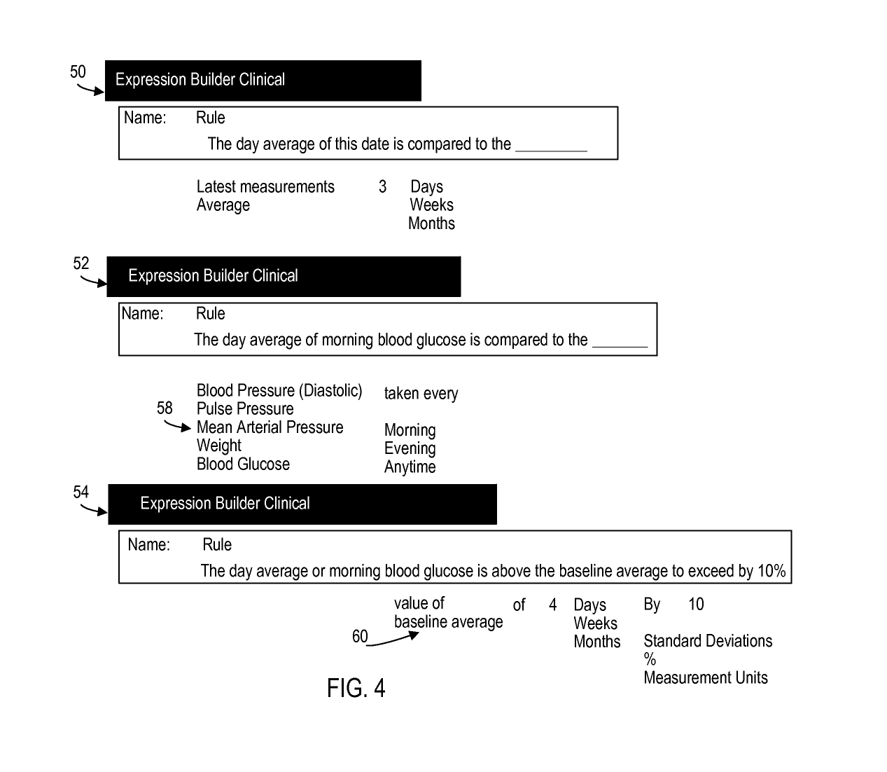

FIG. 4 illustrates an example of interactively programming a rule determining a shift according to the adaptive windowing method and in accordance with certain embodiments. This interactive programming proceeds in three steps. In first step 50, options 56 are interactively presented according to which an operator can select a current window size, here 3 days, and the manner in which the current values are combined, here an average. In step 52, options 58 are presented from which the type of the measurement values can be selected, here morning blood glucose. Finally, in step 54, options 60 are presented from which the baseline window size, here 4 weeks, and the size of the threshold, here 10%, can be selected. The result of selecting among the presented options is a complete adaptive windowing rule for shift, drift or trend detection.

### Event Detection

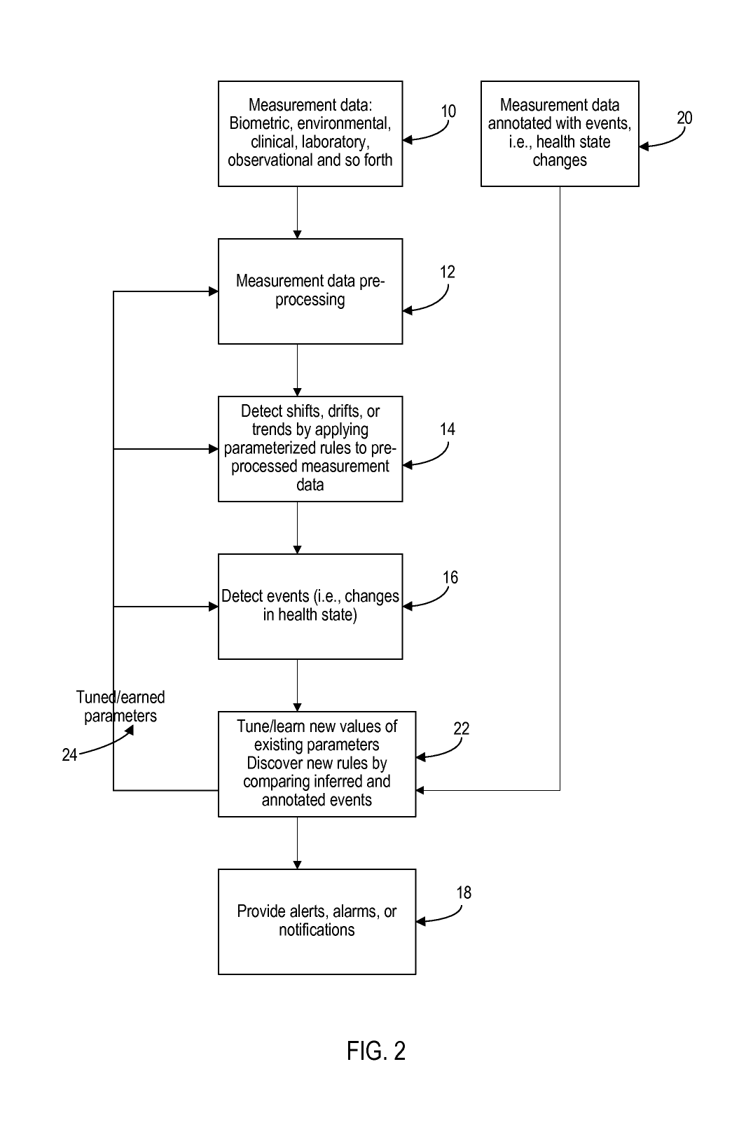

FIG. 2 illustrates another example process that includes rule discovery and/or parameter learning for detecting shifts, drifts and trends in data in accordance with one embodiment. Here, steps 10-18 are as in FIG. 1. What is different from FIG. 1 in this embodiment, is that health state changes, i.e., events, are annotated in the input data in step 20 and are presented for tuning/learning/discovering in step 22. Event annotation can result from a variety of means, including direct manual or automated annotation in the system, or manual or automated data mining from insurance claims or clinical data bases. Then the system compares the annotated events in the measurement data with the events inferred in step 16 from the same measurement data, and in case of a difference, applies a machine learning technique to tune/learn new parameters for the parameterized rules of step 14 or to discover entirely new rules. It should be noted that steps 12 and 16, as well as step 14, can depend on parameters. The tuned/learned parameters, or the newly discovered rules, are then applied to correctly infer events in step 16 and to provide alerts, alarms and notifications in step 18.

With reference to FIGS. 1 and 2, events, i.e., changes in health state, are detected in view of changes (drifts, trends, or shifts), or the absence thereof, in the pre-processed measurement data as determined via the process described above. Event detection logic can be initial event detection logic, that provides event detection (step 16) prior to any tuning or machine learning. Initial even detection logic can be interactively configured, accepted or programmed by an operator. The operator may draw on experience, intuition and/or evidence to program the initial detection logic. This is typically recorded as a set of rules or heuristics depending on parameters operating on the output of the shift, drift, trend detection of step 14.

During system operation, measurement data annotated with actual health events, step 20 in FIG. 2 can be presented to the system so that it will tune/learn/discover, in step 22, new event detection parameters in step 24. The new event detection parameters can be used to develop new detection logic.

Specification or configuration of the initial detection rules can optionally be done using a formal grammar to describe a subset of the natural (human) language to be used for specification. The formal grammar can consist of a set of production rules that specify which sentences can be expressed by the user. This will consist of a small subset of all possible sentences in the natural language. The grammar allows users to build sentences interactively to describe change detection logic.

However the rules are specified, they should be of a format that can be reduced to a finite number of numerical and/or categorical parameters that can be used to re-construct or specify the exact set of rules. Once the rules are parameterized, any changes to these parameters will change the rule(s) and thus the detection logic. Parameterization of the rules allows for subsequent use of automated algorithms such as machine learning or optimization algorithms to update rule parameters and generate new rules.

The system can be configured to allow for customizable alarms or notifications to be set that are triggered when significant health state changes or measurement data drifts are observed. Triggering of an alarm, event or notification will solicit a response, which in turn will either ignore the detected change or drift and continue to look for further change or drift, modify some of the algorithm or detection parameters, or reset the baseline with a subset of the data or begin acquiring new data to form a new baseline. The notification can result in a custom labelling of patient state, such as the reporting of a graded risk category.

In one embodiment, the health states can have a hierarchical structure, that is, a single more general health state can be logically composed of several more particular health sub-states. Statistical process models such as Hidden Markov Models (HMM's) may be used to model state transitions among the sub-states. During baseline conditions, individual will transition between sub-states with state definite transition probabilities, allowing estimations of the state transition probabilities. The more general states are then defined by the collection of transition probabilities between the sub-states.

Deviations from baseline conditions will result in a statistically significantly different set of estimated transition probabilities that thus reflect a change to a new more general state reflected by changes in the underlying sub-state dynamics or transitional probabilities. Thus changes in more general states are detected by changes in sub-state dynamics or transitional probabilities. This can be used to flag significant changes from baseline.

An example of such hierarchical heath states can be found in sleep. When asleep, an individual transitions between various sleep states, i.e., NREM sleep states 1, 2, and 3 and REM sleep, as determined by electroencephalography, with certain transition probabilities. Normal transitions probabilities characterize a normal overall sleep state. When these normal transition probabilities change, the overall sleep state transitions to an abnormal sleep state. The abnormal overall sleep state may be associated with, ex., an abnormal metabolic, state reflected by shifts, drifts, trends in laboratory measurements. The presence of two abnormalities can increase the confidence in a health diagnostic event.

This example, thereby, illustrates how the system can combine temporarily coordinated changes in different measurement types into an overall diagnostic event.

In a preferred embodiment, groups of related rules are hierarchically structured into what are referred to as disease models. Disease model rules are related in that they reflect the values of measurement variables that are closely related to a particular disease. For example, in the case of congestive heart failure, the measurement variables could include blood pressure, heart rate, respiration rate, weight, fatigueability, difficulty breathing, and so forth. Disease model rules can be in the adaptive windowing format to detect shifts, drift or trends in their measured variables. Such rules can be parameterized by, e.g., window sizes and threshold values. They can be arranged into a hierarchy so that paths of true rules (i.e., rules where specified drifts, shifts or trends are actually detected in the input data) lead to leaves specifying a particular medical risk.

### Parameter Tuning/Learning

With reference to FIG. 2, over time the system can obtain ‘truth data’ in step 20, in the form of annotated measurement data which provides objective feedback about actual health-state changes (i.e., events). This annotated measurement data (i.e., truth data) can be acquired in the form of event labels (i.e., annotations) that label health events of interest in a numerical and/or categorical manner. This annotated measurement data can be entered directly by users of the system. It may also be obtained by data mining of health claims data or electronic medical records to determine reported health changes, doctor's visits, hospital admissions, etc. The reported health changes obtained by data mining are then combined with corresponding measurement data to yield ‘truth data.’

The system can label certain ‘events’ as relevant markers for changes in health state. These events can be hospital admissions, physician visits, interventions such as medication or treatment changes, observations or reported diary events (subjective reports by an individual about their health state), etc. The system can examine actual data to refine the events and can consider earlier times than the labelled events as alternative target events.

Predicted health state changes (or events), performed by the evaluation of the existing event detection rules, can be compared in step 22 against actual events to determine a measure of system ‘accuracy’. This can be reported as a sensitivity, specificity, positive or negative predictive values by examining the true and false positive and negative rates.

Knowledge of the labelled health events can be used to optimize, derive or refine the initial set of detection rules or logic. This labelled data can be used to train a machine learning algorithm to generate a new set of rules and parameters in step 24 according to pre-set sensitivity and specificity trade-off targets. The new rules can be evaluated against the original training data to determine new baselines for evaluation against newly acquired data. The new rule can also be edited, e.g. by a human health care professional to generate a hybrid, human-machine disease model.

All possible parameters, continuous and categorical, that span the entire rule space, or a subset of these, can be considered as ‘features’ for a supervised machine learning system. Various feature selection algorithms can then be used to reduce the possible parameters to a parsimonious set. The set of parameters that is obtained from the feature selection process can be used to construct a new set of rules that is a subset of the span of all possible rules.

Coarse graining of the possible set of parameters can be performed to initially reduce the available computations and choices. Further boundaries can be imposed based on knowledge of what is physiologically reasonable or likely.

A cost or loss function can be created that encapsulates the desired event detection accuracy and trade-off between sensitivity and specificity. Various algorithms can then be used to select features such that the cost or loss function is minimized in some sense.

A feature selection algorithm is considered as the combination of a search technique for proposing new feature subsets, along with an evaluation measure, which scores the different feature subsets. Decision tree algorithms are particularly suited to determine a new rule parameter set; however, the systems and methods described herein are not restricted to these algorithms and also considers the use of any other feature selection algorithms including, but not limited to, penalized regression and it's variants e.g. LASSO, Recursive Feature Elimination algorithm, support vector machines, neural networks, stepwise regression, greedy algorithms, exhaustive search algorithms, simulated annealing, genetic algorithms, targeted projection pursuit, basis pursuit, scatter search, variable neighborhood search, consistency-based feature selection, and correlation-based feature selection.

It is also possible to use unsupervised machine learning techniques to identify events that could be used to train the supervised feature selection or rule generation algorithms.

Once training on historic data is complete, the trained system can recommend new rules or changes to existing rules. The system can thus be thought of as a rule recommendation or discovery engine. A user of the system can be presented an interface where they could accept, reject or edit the recommendations to result in a new set of rules or model against which historic or new incoming measurement data will be evaluated. The presented rules can also be compared against the initial set of rules and differences noted and presented to a user.

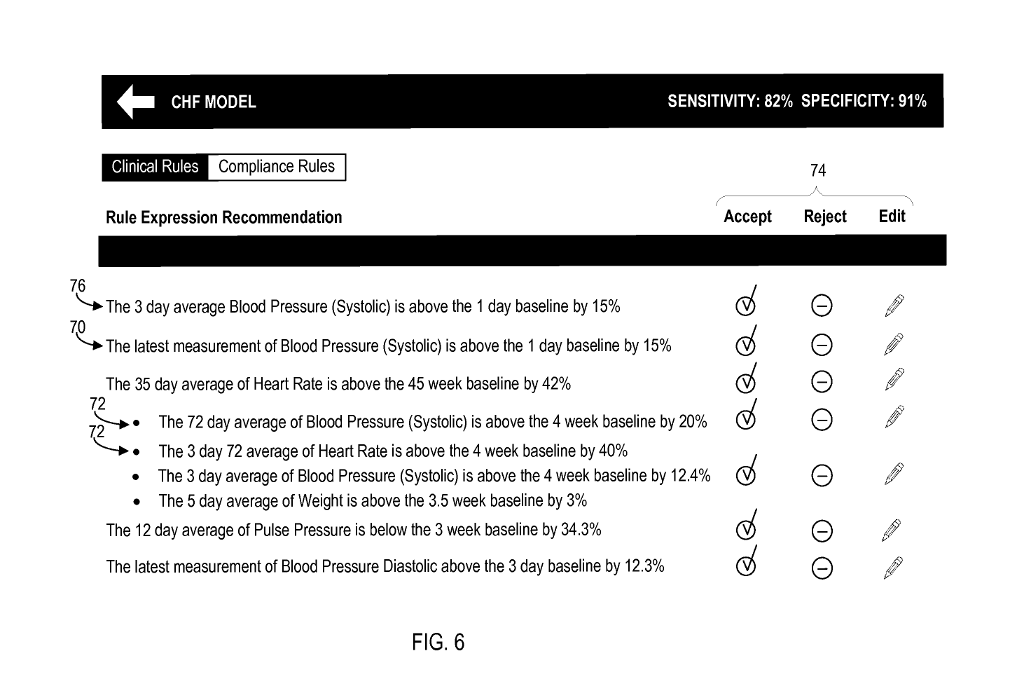

For example, FIG. 6 displays an example of how rules generated by the system can be presented to the user as rule recommendations. This figure illustrates hierarchically grouped rules that relate to congestive heart failure (CHF), which is a model of CHF. The rules, e.g., separate high, medium and low risk states of patients with CHF. The format of each rule is in the adaptive windowing method of shift, drift or trend detection. In this case, the user started with an initial set of rules (shown in black font 76). The system generated new rules and provides a comparison with the initial set of rules. Recommended deletions are indicated by strikeout 70; changes to existing rules are noted in grey font 72; and new rules are also indicated in grey font. Differences may be parameter changes, deletions and/or inclusions to existing rules or to new rules. The user has the option to accept, reject or edit the recommendations 74.

The event labelling or creation of truth data can be performed at discrete intervals or it can be updated in a continuous fashion. Similarly, rule generation or recommendations can be performed at discrete intervals on demand, on customizable triggers or otherwise and can also be performed continuously as each new labelled event is obtained. For large data-sets, online learning implementations of the feature selection algorithms can also be considered versus batch learning implementations.

The set of available patients may be grouped into smaller sub-groups, where a new rule recommendation is assigned to each sub-group. The selection of the sub-groups can be accomplished by several different methods including unsupervised grouping or clustering, segmentation by model performance, or knowledge based grouping.

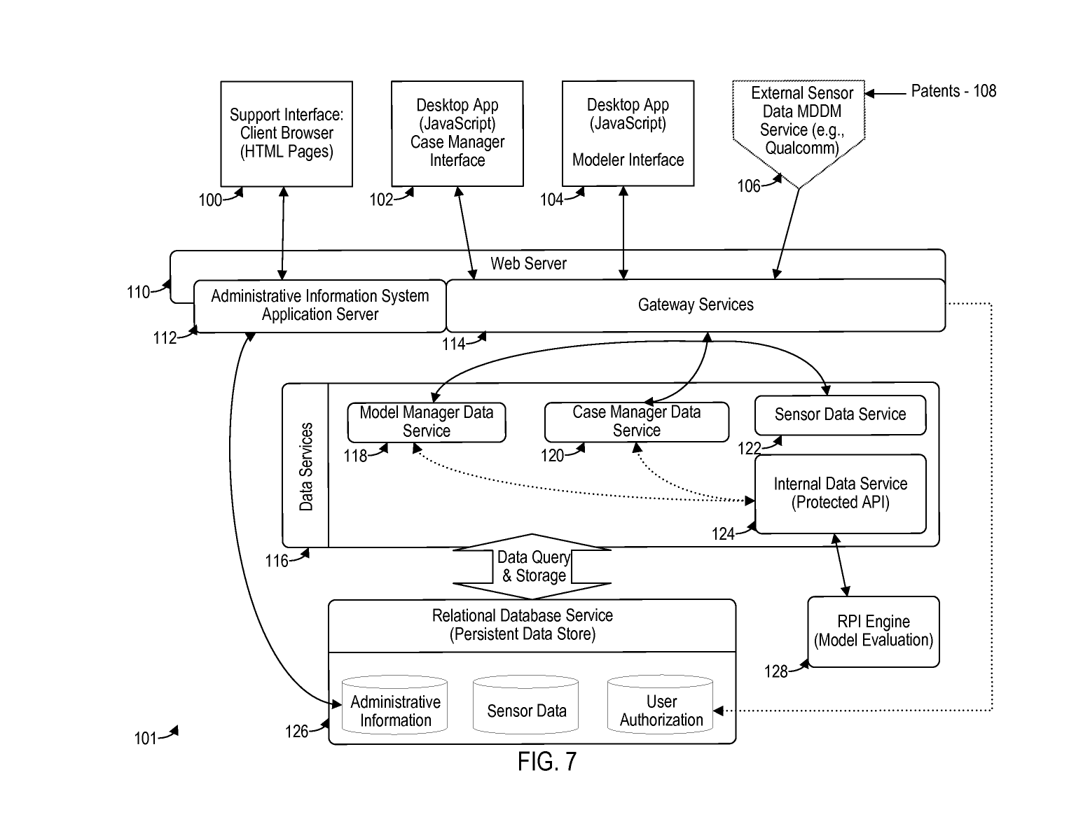

The previously described methods can be performed by systems of various architectures, such as the example architecture illustrated in FIG. 7. System 101 presents several external interfaces by means of a web server 110 and application servers 112 and 114. The external interfaces can include: a support interface 100, a case manager interface 102, and modeler interface 104, and an external sensor medical device data interface 106.

The support interface provides 100 facilities for managing the system configuration, including entry of patient information (e.g. demographics), medical monitoring devices assigned to patients for data collection, and managing users (case managers, modelers, and support staff) who log in to the system. This interface is preferably a web interface encoded by HTML pages and displayed by a standard browser.

The case manager interface 102 provides facilities for case managers, health care professionals who perform the day-to-day patient monitoring. The facilities include the display of patient measurement data values, patient alerts and patient notifications and the capability for directing messages and alerts to the patient. This interface, and the following modeler interface, is preferably a desktop interface encoded in, ex., JavaScript.

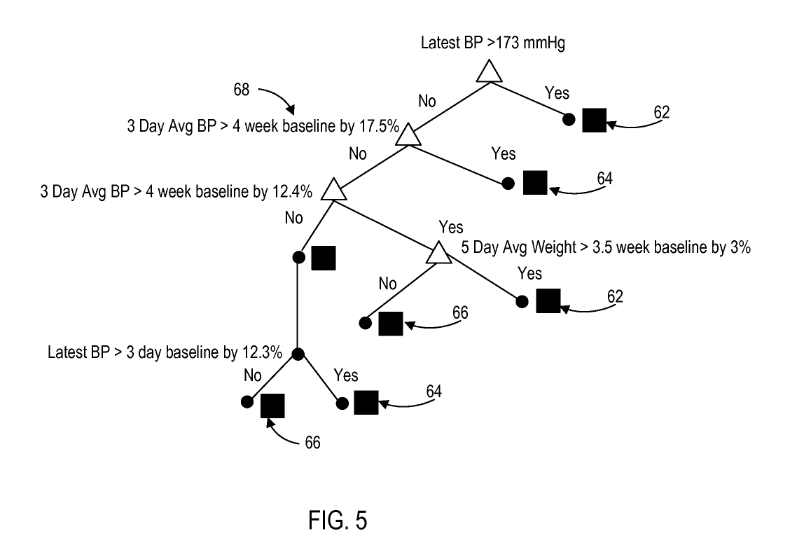

The modeler interface 104 provides facilities for modelers, health care professionals who determine the physiological conditions to be monitored, and what conditions signify attributes of a patient's status that are of interest to the Case Manager. For example, modeler interface 104 can provide facilities for specifying rules, as illustrated in FIG. 4, and for constructing models from a plurality of combined rules as in FIGS. 5 and 6. The modeler interface 104 can also be used to present recommended rules and provide a means for comparison and editing between different disease models.

The external sensor data interface 122 can be configured to receive data from patient sensors 108. The wireless transmission can be provided by third party providers. The patient data can be obtained via wired or wireless transmissions.

Web server 108 can be a standard web server, e.g., an Apache type web server. In cooperation with the administrative application server 112, can serve web pages and data to and from the support interface 100. In cooperation with the gateway services application server 114, can serve data to and from the case manager 102 and modeler interfaces 104. The administrative application server 112 and the gateway services application server 114 can perform such computations as are required to support their frontend interfaces. These components are implemented as is known in the art on network attached server computer with adequate disk storage.

In the case of the external device data interface 106, the web browser can be configured primarily to receive data and to dispatch it to sensor data services 122 in a middleware data services component 116. Among other transformations, sensor data services 122 associates raw data items with the patient and with the sensor type from which they originate so that they can be properly interpreted by subsequent processing. Transformed sensor data can then be stored in relational data base service 126.

Middleware data services component 116 manages various classes of data useful to the system. Model manager data service 118 stores, e.g., rule definitions and model definitions used by the system. Case manager data service 120 stores, e.g., an association between case managers and patients. Sensor data service 122 has already been described. Internal data service 128 is an internal interface that fetches and stores data for the model evaluation engine 128. The middleware data services component 116 can be implemented on network computers with adequate data storage.

Relational or non-relational data base service 126 provide long term (persistent) storage for the types of data illustrated, in particular for administrative information and sensor data. This data is accessible to middleware data services 116 components by means of query and storage commands (e.g., formatted in SQL). These components can be implemented on database system computers.

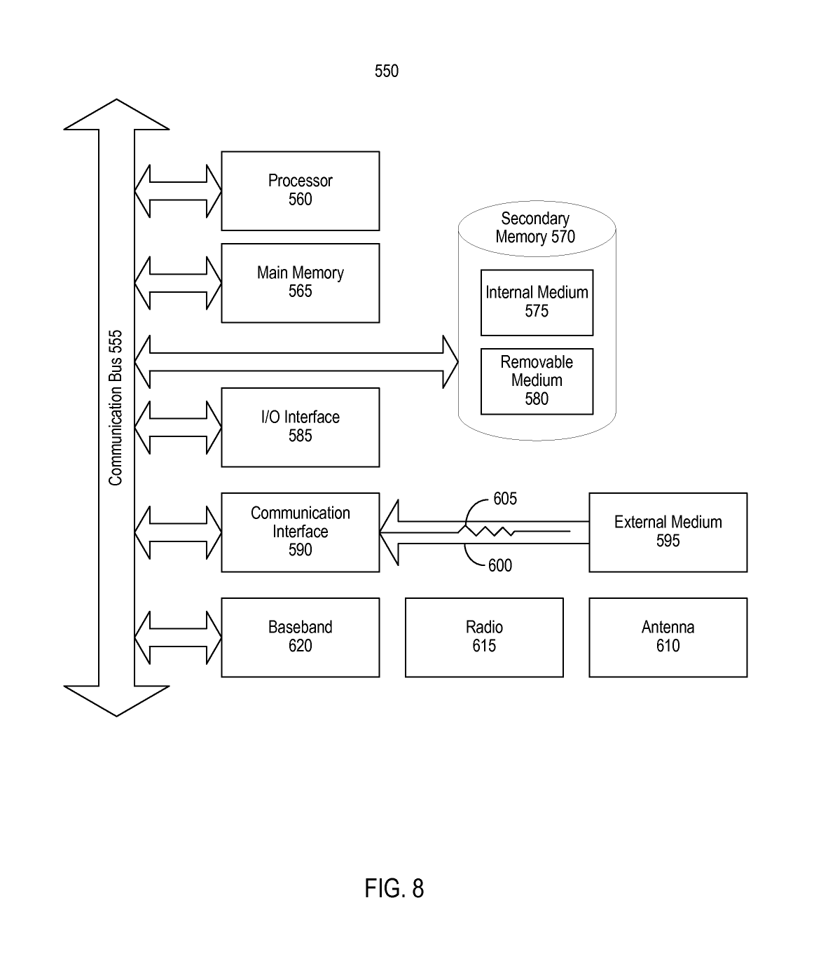

Finally, model evaluation engine 128 can perform rule and model evaluation for the system. It is linked, through internal interface 124, to data services to retrieve rule and model definitions 118 and to sensor data service 122 to retrieve measurement data to which rules and models are applied. It is connected to case manager data service 120 and to other communication services, which are not illustrated to return notification and alerts which result from rule and model processing. Engine 128 also processes rule training and learning. This component can be implemented on a processor of adequate processing power, such as that illustrate in FIG. 8.

The system of FIG. 7 has been described above in terms of separate components and systems implementing these components; however, the system should not so limited. FIG. 7 should be understood as a functional diagram and the functional components illustrated can be implemented on one or more computer systems having network communications and disk storage. Each component can be implemented on its own computer system, or all the components can be implemented in a single system. Alternate partitions of the processing are also possible.

### Example 1

An example of detecting shifts, drifts and trends using system 101 and using the windowing method will be described here. This is generally a preferred method. This method employs a sliding window 30 (FIG. 3) containing the most recently received data points and then compares the distribution on two sub windows of this window. Whenever two large enough sub-windows exhibit statistical significant different averages, an alert or notification is triggered.

A simple implementation of this method compares the averages (mean or median) of the two windows and if the comparison is greater than or less than a selected threshold 36, then an alert is triggered. This threshold can be described as a percentage difference between the two windows.

FIG. 3 illustrates this method in more detail. We consider the 1^{st} moving average window 32 to be a moving baseline period aggregation. The 2^{nd} moving average window 34 is termed a current period aggregation as it is the more recent window relative to the latest received measurement.

For each data point in a time-series, tⱼ, we can describe the average of the current window 36 of n data points as

$${C}_{n} = \frac{1}{n} {\sum }_{i = 1}^{n} {t}_{j - i}$$

Similarly, we can describe the average of the baseline window 38 of m data points as

$${B}_{m} = \frac{1}{m} {\sum }_{i = 1}^{m} {t}_{j - m - i}$$

Here the baseline window is adjacent to the current window.

In this example mean aggregations are used but medians can also be used and medians are preferred for longer baseline durations.

The percentage difference between the two windows for a given data point i can be described as:

$${P}_{i , n , m} = \frac{( {C}_{n} - {B}_{m} )}{{B}_{m}} \times 100$$

Various forms of difference operators are possible.

A threshold parameter, T, may be used to compare against P and if P>T or P<T, the rule may be evaluated to be true, otherwise it is false.

The adaptive windowing method is able to detect trends or shifts and is easily parameterized by 2 duration parameters (n and m) and a third threshold parameter (T). This allows that for every data point a rule can be reduced to a finite set of parameters to describe the rule. This feature allows machine learning of new parameters and, therefore, new instances of the adaptive windowing scheme.

Additional categorical parameters can be introduced for example if the aggregation is a median or mean, then this introduces a fourth parameter that would completely describe the rule.

Further default enhancements to this type of rule can be implemented for example by not including certain points in the aggregation if they are deemed outliers or if they have already generated alerts etc.

These rules can also (optionally) be expressed in natural language e.g. notify me if the 3 day average of some measurement type is 20% greater than the 4 week baseline median

Note that to detect low frequency or slow moving trends, larger window sizes can be used and to detect more abrupt shifts, smaller windows can be employed. For the case of m=0, there is no baseline and this considers comparing the current average against a fixed threshold. If n=1, then this reduces to a simple threshold crossing.

The time series data are typically acquired physiological measurement data. Typically this is a once or twice daily measurement e.g. weight, blood pressure, etc.

For more complex continuous data streams, such as a continuous heart rate (HR), a signal can preprocessed to single daily values, e.g. by counting number of times per day that the HR exceeded 180 bpm etc. This data reduction of continuous data streams results in new derived data streams and rules can be built against these new data streams.

Adaptive windowing implementations evaluate to true or false, and thereby, can be logically combined into combined rules that can better discriminate health events than can single rules.

### Example 2

Presented herein are examples of parameter tuning and learning that can be implemented using system 101. Generating a “feature set” to span the entire adaptive windowing rule space involves specifying maximum ranges for the 2 duration parameters and for the one threshold parameter. One can reasonably bound the durations of each moving window (m and n). Thus baselines between 0 days and 60 days for example are reasonable and current averages between 1 day and 30 days may be considered reasonable.

It is then possible to construct a predictor matrix for each data point that considers all possible aggregation lengths. In this simple two parameter model, we have 30*61=1830 possible combinations for each data point tⱼ

For a 6 month time series of daily measurements, i=180 data points.

For this simple illustration, a predictor matrix (for each patient on the system and for each data series) can be constructed spanning each data point and each possible parameter:

$$X = [ \begin{matrix}{P}_{1 , 1 , 0} & … & {P}_{180 , 1 , 0} \\ ⋮ & ⋱ & ⋮ \\ {P}_{1 , 30 , 60} & … & {P}_{180 , 30 , 60}\end{matrix} ]$$

A response matrix that considers each event class separately can then be constructed. To illustrate, consider a simple 3 class (categorical) model: 0=no event; 1=low risk; 2=high risk. For each patient on the system, we would label all the data in the 6 month as 0 or 1 depending on whether there was a hospital admission.

More complex classes can be considered that look at types of admissions, length of stay and other variables. This system will result in very large predictor matrices but they are still bounded and tractable to subsequent analysis.

With reference to FIG. 5, which illustrates a congestive heart failure (CHF) model, one approach learning a subset of the rules and the thresholds is to use decision tree learning

FIG. 5 represents a classification tree in which leaves represent class labels and branches represent conjunctions of features that lead to those class labels. For example, dark leaf boxes, like box 60, represent high risk medical events or states; gray leaf boxes, like box 62, represent moderate risk medical states; and empty leaf boxes, like box 64, represent low risk medical states. The classifications, which are the risks of various medical states, can be provided to the leaning algorithm as annotations on the input measurement data.

The data upon which the tree is based are shifts, drifts, or trends detected according to the adaptive windowing method. For example, datum 66, “3 Day Avg BP>4 week baseline by 17.5%,” is true if the specified shift is detected and false otherwise. Note that the parameters of this rule are 3 days, 4 weeks, and 17.5%. If this datum is true, the decision tree leads to the moderate risk health event 62. This decision tree leads to high risk leaf node 66 only if combination of rules “3 Day Avg BP>4 week baseline by 12.4%” and “5 Day Avg Weight>3.5 week baseline by 3%” are both true. The data are evaluated directly from the pre-processed input measurement data.

Algorithms for constructing decision trees usually work top-down, by choosing a variable at each step that best splits the set of items. For example, a decision tree algorithm can, in a first instance, chose a variable (such as a shift, drift, or trend in measured data) that best separates high risk medical states from low and moderate risk states, and in a second instance chose a variable that best separates moderate risk medical states from low risk states. Note that the risk of particular states is “truth data” that has been annotated onto the measurement data.

Different algorithms use different metrics for measuring “best”. Concepts such as cost functions that weigh certain rules or parameters to bias the tree toward the importance of these parameters can be introduced into the algorithm. One can also prune trees and consider other tree algorithms such as random forests etc. to improve performance.

In the general implementation, the output of the decision tree learner at each node is a threshold.

Multiple decision trees can also be considered where the result on each decision tree are combined as a logical OR. i.e. if any decision tree results in a true state, then the result of the system is true.

Thus from FIG. 5, we have two time series, weight and BP. The root node has the baseline m=0 and the current window length m=1. The algorithm solved for a threshold on BP of 173 and this results in a class label 2=high risk.

The 2^{nd} node is m=3, n=28, BP data stream and a threshold of 17.5%

Combined rules are given by conjunctions in the tree. Thus left hand branches can be considered as individual rules that are joined by OR logic and right hand branches as combined rules joined by AND logic. The system can allow multiple conjunctions and can thus be used to discover patterns not easily discernable to human users that set the initial rules.

Thus, shift, drift, trend or outlier detection rules that operate on longitudinal measurements can be parameterized; Annotated historical time series data can then be used to generate a set of rules that best detect the labels in some optimal sense. Decision trees are a very good example of how this learning can be achieved but the systems and methods described herein should not restricted to decision trees. Other sparse feature reduction algorithms can be used to reduce a very large set of possible parameters (i.e. rules) to a smaller subset.

FIG. 8 is a block diagram illustrating an example wired or wireless system 550 that can be used in connection with various embodiments described herein. For example the system 550 can be used as or in conjunction with one or more of the mechanisms or processes described above, and may represent components of system 100, the corresponding backend server(s), and/or other devices described herein. The system 550 can be a combination of one or more of the following: a server or any conventional personal computer, or any other processor-enabled device that is capable of wired or wireless data communication. Other computer systems and/or architectures may be also used, as will be clear to those skilled in the art.

The system 550 preferably includes one or more processors, such as processor 560. Additional processors may be provided, such as an auxiliary processor to manage input/output, an auxiliary processor to perform floating point mathematical operations, a special-purpose microprocessor having an architecture suitable for fast execution of signal processing algorithms (e.g., digital signal processor), a slave processor subordinate to the main processing system (e.g., back-end processor), an additional microprocessor or controller for dual or multiple processor systems, or a coprocessor. Such auxiliary processors may be discrete processors or may be integrated with the processor 560. Examples of processors which may be used with system 550 include, without limitation, the Pentium® processor, Core i7® processor, and Xeon® processor, all of which are available from Intel Corporation of Santa Clara, Calif.

The processor 560 is preferably connected to a communication bus 555. The communication bus 555 may include a data channel for facilitating information transfer between storage and other peripheral components of the system 550. The communication bus 555 further may provide a set of signals used for communication with the processor 560, including a data bus, address bus, and control bus (not shown). The communication bus 555 may comprise any standard or non-standard bus architecture such as, for example, bus architectures compliant with industry standard architecture (ISA), extended industry standard architecture (EISA), Micro Channel Architecture (MCA), peripheral component interconnect (PCI) local bus, or standards promulgated by the Institute of Electrical and Electronics Engineers (IEEE) including IEEE 488 general-purpose interface bus (GPIB), IEEE 696/S-100, and the like.

System 550 preferably includes a main memory 565 and may also include a secondary memory 570. The main memory 565 provides storage of instructions and data for programs executing on the processor 560, such as one or more of the functions and/or modules discussed above. It should be understood that programs stored in the memory and executed by processor 560 may be written and/or compiled according to any suitable language, including without limitation C/C++, Java, JavaScript, Pearl, Visual Basic, .NET, and the like. The main memory 565 is typically semiconductor-based memory such as dynamic random access memory (DRAM) and/or static random access memory (SRAM). Other semiconductor-based memory types include, for example, synchronous dynamic random access memory (SDRAM), Rambus dynamic random access memory (RDRAM), ferroelectric random access memory (FRAM), and the like, including read only memory (ROM).

The secondary memory 570 may optionally include an internal memory 575 and/or a removable medium 580, for example a floppy disk drive, a magnetic tape drive, a compact disc (CD) drive, a digital versatile disc (DVD) drive, other optical drive, a flash memory drive, etc. The removable medium 580 is read from and/or written to in a well-known manner. Removable storage medium 580 may be, for example, a floppy disk, magnetic tape, CD, DVD, SD card, etc.

The removable storage medium 580 is a non-transitory computer-readable medium having stored thereon computer executable code (i.e., software) and/or data. The computer software or data stored on the removable storage medium 580 is read into the system 550 for execution by the processor 560.

In alternative embodiments, secondary memory 570 may include other similar means for allowing computer programs or other data or instructions to be loaded into the system 550. Such means may include, for example, an external storage medium 595 and an interface 590. Examples of external storage medium 595 may include an external hard disk drive or an external optical drive, or and external magneto-optical drive.

Other examples of secondary memory 570 may include semiconductor-based memory such as programmable read-only memory (PROM), erasable programmable read-only memory (EPROM), electrically erasable read-only memory (EEPROM), or flash memory (block oriented memory similar to EEPROM). Also included are any other removable storage media 580 and communication interface 590, which allow software and data to be transferred from an external medium 595 to the system 550.

System 550 may include a communication interface 590. The communication interface 590 allows software and data to be transferred between system 550 and external devices (e.g. printers), networks, or information sources. For example, computer software or executable code may be transferred to system 550 from a network server via communication interface 590. Examples of communication interface 590 include a built-in network adapter, network interface card (NIC), Personal Computer Memory Card International Association (PCMCIA) network card, card bus network adapter, wireless network adapter, Universal Serial Bus (USB) network adapter, modem, a network interface card (NIC), a wireless data card, a communications port, an infrared interface, an IEEE 1394 fire-wire, or any other device capable of interfacing system 550 with a network or another computing device.

Communication interface 590 preferably implements industry promulgated protocol standards, such as Ethernet IEEE 802 standards, Fiber Channel, digital subscriber line (DSL), asynchronous digital subscriber line (ADSL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and so on, but may also implement customized or non-standard interface protocols as well.

Software and data transferred via communication interface 590 are generally in the form of electrical communication signals 605. These signals 605 are preferably provided to communication interface 590 via a communication channel 600. In one embodiment, the communication channel 600 may be a wired or wireless network, or any variety of other communication links. Communication channel 600 carries signals 605 and can be implemented using a variety of wired or wireless communication means including wire or cable, fiber optics, conventional phone line, cellular phone link, wireless data communication link, radio frequency (“RF”) link, or infrared link, just to name a few.

Computer executable code (i.e., computer programs or software) is stored in the main memory 565 and/or the secondary memory 570. Computer programs can also be received via communication interface 590 and stored in the main memory 565 and/or the secondary memory 570. Such computer programs, when executed, enable the system 550 to perform the various functions of the present invention as previously described.

In this description, the term “computer readable medium” is used to refer to any non-transitory computer readable storage media used to provide computer executable code (e.g., software and computer programs) to the system 550. Examples of these media include main memory 565, secondary memory 570 (including internal memory 575, removable medium 580, and external storage medium 595), and any peripheral device communicatively coupled with communication interface 590 (including a network information server or other network device). These non-transitory computer readable mediums are means for providing executable code, programming instructions, and software to the system 550.

In an embodiment that is implemented using software, the software may be stored on a computer readable medium and loaded into the system 550 by way of removable medium 580, I/O interface 585, or communication interface 590. In such an embodiment, the software is loaded into the system 550 in the form of electrical communication signals 605. The software, when executed by the processor 560, preferably causes the processor 560 to perform the inventive features and functions previously described herein.

In an embodiment, I/O interface 585 provides an interface between one or more components of system 550 and one or more input and/or output devices. Example input devices include, without limitation, keyboards, touch screens or other touch-sensitive devices, biometric sensing devices, computer mice, trackballs, pen-based pointing devices, and the like. Examples of output devices include, without limitation, cathode ray tubes (CRTs), plasma displays, light-emitting diode (LED) displays, liquid crystal displays (LCDs), printers, vacuum florescent displays (VFDs), surface-conduction electron-emitter displays (SEDs), field emission displays (FEDs), and the like.

The system 550 also includes optional wireless communication components that facilitate wireless communication over a voice and over a data network. The wireless communication components comprise an antenna system 610, a radio system 615 and a baseband system 620. In the system 550, radio frequency (RF) signals are transmitted and received over the air by the antenna system 610 under the management of the radio system 615.

In one embodiment, the antenna system 610 may comprise one or more antennae and one or more multiplexors (not shown) that perform a switching function to provide the antenna system 610 with transmit and receive signal paths. In the receive path, received RF signals can be coupled from a multiplexor to a low noise amplifier (not shown) that amplifies the received RF signal and sends the amplified signal to the radio system 615.

In alternative embodiments, the radio system 615 may comprise one or more radios that are configured to communicate over various frequencies. In one embodiment, the radio system 615 may combine a demodulator (not shown) and modulator (not shown) in one integrated circuit (IC). The demodulator and modulator can also be separate components. In the incoming path, the demodulator strips away the RF carrier signal leaving a baseband receive audio signal, which is sent from the radio system 615 to the baseband system 620.

If the received signal contains audio information, then baseband system 620 decodes the signal and converts it to an analog signal. Then the signal is amplified and sent to a speaker. The baseband system 620 also receives analog audio signals from a microphone. These analog audio signals are converted to digital signals and encoded by the baseband system 620. The baseband system 620 also codes the digital signals for transmission and generates a baseband transmit audio signal that is routed to the modulator portion of the radio system 615. The modulator mixes the baseband transmit audio signal with an RF carrier signal generating an RF transmit signal that is routed to the antenna system and may pass through a power amplifier (not shown). The power amplifier amplifies the RF transmit signal and routes it to the antenna system 610 where the signal is switched to the antenna port for transmission.

The baseband system 620 is also communicatively coupled with the processor 560. The central processing unit 560 has access to data storage areas 565 and 570. The central processing unit 560 is preferably configured to execute instructions (i.e., computer programs or software) that can be stored in the memory 565 or the secondary memory 570. Computer programs can also be received from the baseband processor 610 and stored in the data storage area 565 or in secondary memory 570, or executed upon receipt. Such computer programs, when executed, enable the system 550 to perform the various functions of the present invention as previously described. For example, data storage areas 565 may include various software modules (not shown).

Various embodiments may also be implemented primarily in hardware using, for example, components such as application specific integrated circuits (ASICs), or field programmable gate arrays (FPGAs). Implementation of a hardware state machine capable of performing the functions described herein will also be apparent to those skilled in the relevant art. Various embodiments may also be implemented using a combination of both hardware and software.

Furthermore, those of skill in the art will appreciate that the various illustrative logical blocks, modules, circuits, and method steps described in connection with the above described figures and the embodiments disclosed herein can often be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a module, block, circuit or step is for ease of description. Specific functions or steps can be moved from one module, block or circuit to another without departing from the invention.

Moreover, the various illustrative logical blocks, modules, functions, and methods described in connection with the embodiments disclosed herein can be implemented or performed with a general purpose processor, a digital signal processor (DSP), an ASIC, FPGA or other programmable logic device, discrete gate or transistor logic, discrete hardware components, or any combination thereof designed to perform the functions described herein. A general-purpose processor can be a microprocessor, but in the alternative, the processor can be any processor, controller, microcontroller, or state machine. A processor can also be implemented as a combination of computing devices, for example, a combination of a DSP and a microprocessor, a plurality of microprocessors, one or more microprocessors in conjunction with a DSP core, or any other such configuration.

Additionally, the steps of a method or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of storage medium including a network storage medium. An exemplary storage medium can be coupled to the processor such that the processor can read information from, and write information to, the storage medium. In the alternative, the storage medium can be integral to the processor. The processor and the storage medium can also reside in an ASIC.

Any of the software components described herein may take a variety of forms. For example, a component may be a stand-alone software package, or it may be a software package incorporated as a “tool” in a larger software product. It may be downloadable from a network, for example, a website, as a stand-alone product or as an add-in package for installation in an existing software application. It may also be available as a client-server software application, as a web-enabled software application, and/or as a mobile application.

While certain embodiments have been described above, it will be understood that the embodiments described are by way of example only. Accordingly, the systems and methods described herein should not be limited based on the described embodiments. Rather, the systems and methods described herein should only be limited in light of the claims that follow when taken in conjunction with the above description and accompanying drawings.

What is claimed is:

1. A health-state detection system comprising:
a communications interface system for receiving at least one type of medical measurement data pertaining to one or more patients;
a care manager interface system for communicating with one or more care managers of the patients;
a user interface configured to receive one or more rules related to each type of medical measurement data and that define changes in health state;
one or more processors programmed to:
parametrize the rules to generate a set of parameterized rules that define shifts, drifts and trends in the medical measurement data,
determine a baseline for each type of medical measurement data,
execute the parameterized rules for newly received medical measurement data against the baseline for each type of medical measurement data to detect shifts, drifts or trends in the medical measurement data,
determine the magnitudes and directions of the detected shifts, drifts or trends,
correlate the determined magnitudes and directions of the shifts, drifts, or trends detected with changes in the health state of one or more patients, and
provide customizable notifications, alerts or alarms to care managers over the care manager interface system based at least in the past on the detected health state changes.

2. The system of claim 1, further comprising a measurement database storing historic medical measurement data received over the communication interface system.

3. The system of claim 1, wherein the types of medical measurement data include one or more of biometric data, clinical data, laboratory data, environmental data, and observation data.

4. The system of claim 1, wherein parameterized rules comprise a first baseline window parameter, a first current window parameter, and a threshold parameter, and wherein execution of the parameterized rule causes the processor to detect shifts, drifts or trends in medical measurement data of a particular type for a particular patient by:
analyzing a first earlier portion of the medical measurement data that corresponds to the first baseline window parameter to derive a first baseline measurement value,
analyzing a later second portion of the medical measurement data that corresponds to the first current window parameter to derive a first current measurement value, wherein the current window spans a time period that is subsequent to a time period associated with the baseline window, and
detecting a shift, drift or trend if the first current measurement value is significantly different from the first baseline measurement value according to the threshold parameter.

5. The system of claim 1, wherein the baseline for each type of medical measurement data is updated as new medical measurement data are received.

6. The system of claim 1, wherein a changed health state of a patient is detected if shifts, drifts or trends are detected in the medical measurement data that are indicative of a new or changed health state.

7. The system of claim 1, wherein the processor is further configured to combine the rules for a plurality of types of medical measurement data to generate a disease model.

8. The system of claim 7, wherein the disease model is for congestive heart failure, and wherein rules are combined for at least some of the following types of medical measurement data: blood pressure, pulse rate, respiration, and recent weight gain.

9. The system of claim 7, wherein the rules are hierarchically arranged in a tree so that if the particular shifts, drifts or trends to be detected by the rules along a path through the tree from the root to a leaf are actually detected, then the disease associated with the disease model is detected.

10. The system of claim 9, wherein the hierarchical arrangement is a decision tree.

11. The system of claim 3, wherein the measurement database further stores annotations of actual health state changes in the one or more patients, and wherein the processor is further configured to update rule parameters so that the actual annotated health state changes correspond to the health state changes detected by execution of the parameterized rules.

12. The system of claim 11, wherein the processor updates sets of rule parameters selected by a feature selection procedure, wherein the feature selection procedure further includes a search technique for discovering new sets of rule parameters, and an evaluation technique that ranks new sets of rule parameters in terms of their ability to explain the annotated health state changes.

13. The system of claim 12, wherein the updated rule parameters are presented to a user of the system via the user interface so that they may be accepted, rejected, or edited.

14. A health-state detection method, comprising:
receiving at least one type of medical measurement data pertaining to one or more patients;
receiving one or more rules related to each type of medical measurement data and that define changes in health state;
parametrizing the rules to generate a set of parameterized rules that define shifts, drifts and trends in the medical measurement data,
determining a baseline for each type of medical measurement data,
executing the parameterized rules for newly received medical measurement data against the baseline for each type of medical measurement data to detect shifts, drifts or trends in the medical measurement data,
determining the magnitudes and directions of the detected shifts, drifts or trends,
correlating the determined magnitudes and directions of the shifts, drifts, or trends detected with changes in the health state of one or more patients, and
providing customizable notifications, alerts or alarms to care managers over the care manager interface system based at least in the past on the detected health state changes.

15. The method of claim 14, further comprising storing historic medical measurement data received over the communication interface system.

16. The method of claim 14, wherein the types of medical measurement data include one or more of biometric data, clinical data, laboratory data, environmental data, and observation data.

17. The method of claim 14, wherein parameterized rules comprise a first baseline window parameter, a first current window parameter, and a threshold parameter, and wherein executing the parameterized rule detects shifts, drifts or trends in medical measurement data of a particular type for a particular patient by:
analyzing a first earlier portion of the medical measurement data that corresponds to the first baseline window parameter to derive a first baseline measurement value,
analyzing a later second portion of the medical measurement data that corresponds to the first current window parameter to derive a first current measurement value, wherein the current window spans a time period that is subsequent to a time period associated with the baseline window, and
detecting a shift, drift or trend if the first current measurement value is significantly different from the first baseline measurement value according to the threshold parameter.

18. The method of claim 14, further comprising updating the baseline for each type of medical measurement data as new medical measurement data are received.

19. The method of claim 14, wherein a changed health state of a patient is detected if shifts, drifts or trends are detected in the medical measurement data that are indicative of a new or changed health state.

20. The method of claim 14, further comprising combining the rules for a plurality of types of medical measurement data to generate a disease model.

21. The method of claim 20, wherein the disease model is for congestive heart failure, and wherein rules are combined for at least some of the following types of medical measurement data: blood pressure, pulse rate, respiration, and recent weight gain.

22. The method of claim 20, wherein the rules are hierarchically arranged in a tree so that if the particular shifts, drifts or trends to be detected by the rules along a path through the tree from the root to a leaf are actually detected, then the disease associated with the disease model is detected.

23. The method of claim 22, wherein the hierarchical arrangement is a decision tree.

24. The method of claim 15, further comprising storing annotations of actual health state changes in the one or more patients, and updating rule parameters so that the actual annotated health state changes correspond to the health state changes detected by execution of the parameterized rules.

25. The method of claim 24, further comprising updating sets of rule parameters selected by a feature selection procedure, wherein the feature selection procedure further includes a search technique for discovering new sets of rule parameters, and an evaluation technique that ranks new sets of rule parameters in terms of their ability to explain the annotated health state changes.

26. The method of claim 25, further comprising presenting the updated rule parameters to a user of the system via the user interface so that they may be accepted, rejected, or edited.
